(19) United States
(12) Patent Application Publication
(10) Pub. No.: US 2026/0182879 A1
(43) Pub. Date: Jul. 2, 2026

(54) Mental Disorder Analysis Apparatus

(71) Applicant: I'MBESIDEYOU INC., Tokyo (JP)

(72) Inventors: Shozo KAMIYA, Tokyo (JP); Kotaro Ando, Tokyo (JP)

(21) Appl. No.: 19/416,688

(22) Filed: Dec. 11, 2025

(30) Foreign Application Priority Data
Dec. 26, 2024 (JP) 2024-229606

(51) Int. Cl. A61B 5/16; A61B 5/00

(52) U.S. Cl. CPC A61B 5/165; A61B 5/72; A61B 5/742

(57) ABSTRACT

Technical Problem: To provide an apparatus for analyzing whether a person has a mental disorder. Solution to Problem: The system of the present disclosure comprises: a storage unit that stores a mental disorder learning model trained using training videos of patients diagnosed with mental disorders as teaching data; an acquisition unit that acquires an analysis target video containing an analysis target person; and an analysis unit that analyzes whether the analysis target person has the mental disorder by applying the mental disorder learning model to the analysis target video.

### TECHNICAL FIELD

The present invention relates to a mental disorder analysis apparatus.

### BACKGROUND OF THE INVENTION

Technology for analyzing emotions that others receive in response to a speaker's utterance is known (for example, see PTL 1). In addition, technology for analyzing changes in the facial expressions of a target person over a long period of time in chronological order and estimating the emotions felt during that period is also known (for example, see PTL 2). Furthermore, technology for identifying the elements that most influenced changes in emotions is also known (for example, see PTL 3 to 5). Moreover, technology for issuing an alert when a facial expression is dark by comparing the target person's usual facial expression with their current facial expression is also known (for example, see PTL 6). Furthermore, technology that determines the degree of a target person's emotions by comparing the target person's normal (expressionless) facial expression with their current facial expression is also known (for example, see PTL 7 to 9). Additionally, technology for analyzing emotions as an organization and the atmosphere an individual feels within a group is also known (for example, see PTL 10, 11).

### PRIOR ART

[PTL1] Japanese Unexamined Patent Application Publication No. 2019-58625

[PTL2] Japanese Unexamined Patent Application Publication No. 2016-149063

[PTL3] Japanese Unexamined Patent Application Publication No. 2020-86559

[PTL4] Japanese Unexamined Patent Application Publication No. 2000-76421

[PTL5] Japanese Unexamined Patent Application Publication No. 2017-201499

[PTL6] Japanese Unexamined Patent Application Publication No. 2018-112831

[PTL7] Japanese Unexamined Patent Application Publication No. 2011-154665

[PTL8] Japanese Unexamined Patent Application Publication No. 2012-8949

[PTL9] Japanese Unexamined Patent Application Publication No. 2013-300

[PTL10] Japanese Unexamined Patent Application Publication No. 2011-186521

[PTL11] International Publication No. WO15/174426

### SUMMARY OF THE INVENTION

All of the technologies mentioned above can analyze a target person from multiple angles, but they cannot estimate a specific state. In particular, improvements are needed to determine whether a person is in a state related to a disease.

The present invention aims to make it possible to determine whether an analysis target person is in a state related to a mental disorder.

According to the present invention, a mental disorder analysis apparatus is obtained, the apparatus comprising:

a storage unit that stores a mental disorder learning model trained using training videos of patients diagnosed with mental disorders as teaching data;

an acquisition unit that acquires an analysis target video containing an analysis target person; and

an analysis unit that analyzes whether the analysis target person has the mental disorder by applying the mental disorder learning model to the analysis target video.

According to the present disclosure, the burden of medical examination related to the determination of mental disorders can be reduced.

### BRIEF DESCRIPTION OF DRAWINGS

FIG. 1 is a diagram showing the overall system according to an embodiment of the present invention.

FIG. 2 is an example of a functional block diagram according to an embodiment of the present invention.

FIG. 3 is a diagram showing an example of the functional configuration of a video analysis apparatus according to an embodiment of the present invention.

FIG. 4 is a diagram showing an example of the functional configuration of a mental disorder analysis apparatus according to an embodiment of the present invention.

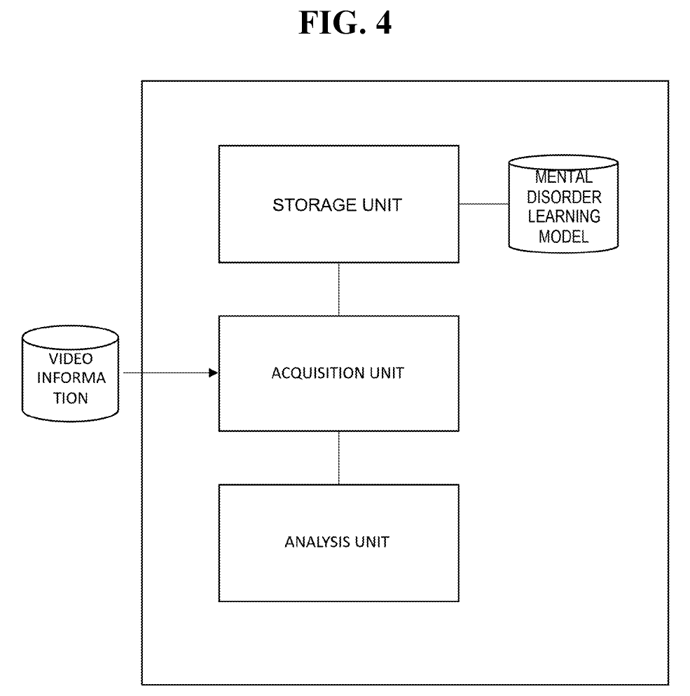

FIG. 5 is a diagram showing the flow of the mental disorder apparatus.

FIG. 6 is a diagram showing the flow of the mental disorder apparatus.

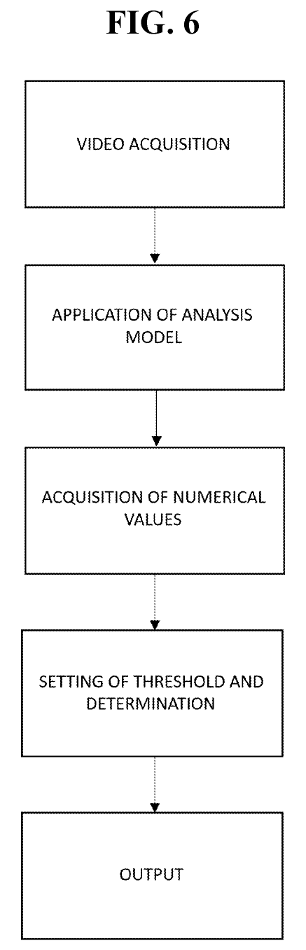

### DETAILED DESCRIPTION OF THE INVENTION

The content of the embodiments of the present disclosure will be described by listing. The present disclosure includes the following configurations.

### [Item 1]

A mental disorder analysis apparatus comprising:

a storage unit that stores a mental disorder learning model trained using training videos of patients diagnosed with mental disorders as teaching data;

an acquisition unit that acquires an analysis target video containing an analysis target person; and

an analysis unit that analyzes whether the analysis target person has the mental disorder by applying the mental disorder learning model to the analysis target video.

### [Item 2]

The mental disorder analysis apparatus according to item 1, wherein

the training videos are recordings of both a first subject and a second subject when they are undergoing a diagnostic process for the mental disorder by an expert,

the first subject is a person who has been previously diagnosed with the mental disorder, and the second subject is a person who has not been previously diagnosed with the mental disorder.

### [Item 3]

The mental disorder analysis apparatus according to item 1, wherein

the analysis target video includes at least the facial expressions and voice of the analysis target person.

### [Item 4]

The mental disorder analysis apparatus according to item 1, wherein

the analysis target video relates to a conversation that the target person is having with another person.

### [Item 5]

The mental disorder analysis apparatus according to item 1, wherein

the analysis target video is obtained using an online session.

### [Item 6]

The mental disorder analysis apparatus according to item 1, wherein

the analysis unit includes information regarding whether the degree of the mental disorder of the analysis target person exceeds a predetermined threshold.

### [Item 7]

The mental disorder analysis apparatus according to item 1, further comprising:

a question unit that asks predetermined additional questions to the analysis target person; and

an answer acquisition unit that acquires responses to the additional questions,

wherein the analysis unit analyzes whether the analysis target person has the specific mental disorder by analyzing the responses.

### [Item 8]

A mental disorder analysis method comprising:

storing, in a storage unit of a computer, a mental disorder learning model trained using training videos of patients diagnosed with mental disorders as teaching data;

acquiring an analysis target video containing an analysis target person; and

analyzing whether the analysis target person has the mental disorder by applying the mental disorder learning model to the analysis target video.

### [Item 9]

A mental disorder analysis system comprising:

a storage unit that stores a mental disorder learning model trained using training videos of patients diagnosed with mental disorders as teaching data;

an acquisition unit that acquires an analysis target video containing an analysis target person; and

an analysis unit that analyzes whether the analysis target person has the mental disorder by applying the mental disorder learning model to the analysis target video.

### [Item 10]

A mental disorder analysis program causing a computer to function as:

a storage unit that stores a mental disorder learning model trained using training videos of patients diagnosed with mental disorders as teaching data;

an acquisition unit that acquires an analysis target video containing an analysis target person; and

an analysis unit that analyzes whether the analysis target person has the mental disorder by applying the mental disorder learning model to the analysis target video.

In the following, preferred embodiments of the present disclosure will be described in detail with reference to the accompanying drawings. In this specification and drawings, overlapping explanations are omitted by assigning the same reference numerals to components having substantially the same functional configuration.

The present invention relates to a system that analyzes videos recording the communication behavior of an analysis target person and analyzes whether the person is in a specific mental state (for example, mental disorders can be exemplified, but not limited to these).

This system generates a mental disorder learning model trained using training videos of patients diagnosed with mental disorders as teaching data, and analyzes whether an analysis target person has a mental disorder by applying the mental disorder learning model to videos containing the analysis target person. The generation of the mental disorder learning model and the analysis of the analysis target person are performed using the video analysis apparatus described later.

The following describes embodiments of this system in the following order.

1. Video Analysis Apparatus

Basic functions of the video analysis apparatus

Hardware configuration example

Method of acquiring videos

Analysis flow

2. Model Generation

Overview

Model generation technique

Other model generation methods

3. Analysis of the Analysis Target Person

Acquisition of videos

Application of the analysis model

Obtaining numerical values

Setting thresholds and determination

Output format

Analysis target examples

### 1. Video Analysis Apparatus

### Basic Functions

The video analysis apparatus of this embodiment is a system that analyzes and evaluates specific emotions (feelings that occur in response to one's own or others' words and actions, such as pleasure, displeasure, or their degree) of an analysis target person among multiple people in an environment where video sessions (hereinafter referred to as online sessions, including both one-way and two-way) are conducted. Online sessions include, for example, online meetings, online classes, online chats, etc., which connect terminals installed in multiple locations to a server via communication networks such as the Internet, enabling the exchange of video images between multiple terminals through the server. The video images handled in online sessions include facial images and voices of users using the terminals. The video images also include images of materials that multiple users share and view. On the screen of each terminal, it is possible to switch between facial images and material images to display only one of them, or to divide the display area to display facial images and material images simultaneously. It is also possible to display one person's image in full screen, or to divide the screen into small sections to display images of some or all users. Among multiple users participating in an online session using terminals, it is possible to designate one or more people as analysis target persons. For example, the leader, facilitator, or administrator (hereinafter collectively referred to as the host) of the online session designates a user as an analysis target person. The host of an online session is, for example, an instructor of an online class, a chairperson or facilitator of an online meeting, a coach of a session for coaching purposes, etc. The host of an online session is usually one of the multiple users participating in the online session, but may also be a different person who does not participate in the online session. It is also possible not to designate an analysis target person and to analyze all participants. In addition, the leader, facilitator, or administrator (hereinafter collectively referred to as the host) of the online session can designate a user as an analysis target person. The host of an online session is, for example, an instructor of an online class, a chairperson or facilitator of an online meeting, a coach of a session for coaching purposes, etc. The host of an online session is usually one of the multiple users participating in the online session, but may also be a different person who does not participate in the online session.

The video analysis apparatus according to this embodiment displays at least video images obtained from the video session when a video session is established between multiple terminals. The displayed video images are acquired by the terminal, and at least facial images included in the video images are identified for each predetermined frame unit. Subsequently, evaluation values related to the identified facial images are calculated. These evaluation values are shared as needed. In particular, in this embodiment, the acquired video images are stored in the terminal, analyzed and evaluated on the terminal, and the results are provided to the user of the terminal. Therefore, even for video sessions containing personal information or confidential information, analysis and evaluation can be performed without providing the video itself to external evaluation organizations. Also, if necessary, by providing only the evaluation results (evaluation values) to external terminals, the results can be visualized or cross-analyzed.

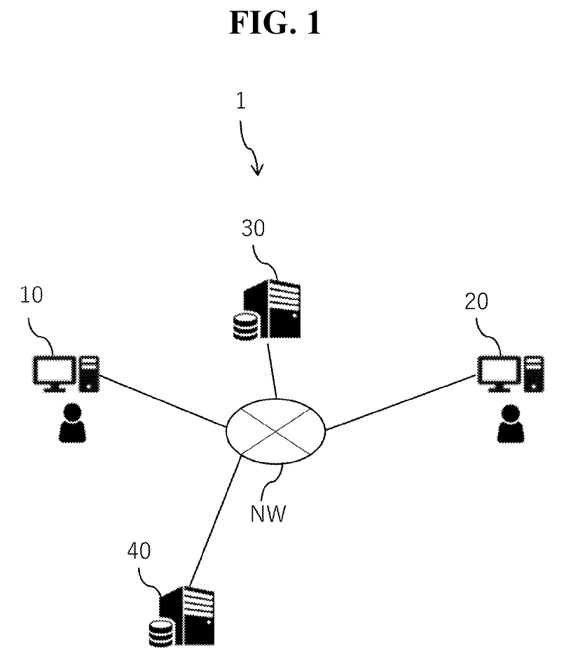

As shown in FIG. 1, the video analysis apparatus according to this embodiment comprises user terminals 10, 20 having at least an input unit such as a camera unit and a microphone unit, a display unit such as a display, and an output unit such as a speaker; a video session service terminal 30 that provides two-way video sessions to the user terminals 10, 20; and an evaluation terminal 40 that performs part of the evaluation related to the video sessions.

### Hardware Configuration Example

Each functional block, functional unit, and functional module described below can be configured by hardware equipped in a computer, a DSP (Digital Signal Processor), or software. For example, when configured by software, it is actually composed of a computer's CPU, RAM, ROM, etc., and is realized by the operation of a program stored in a recording medium such as RAM, ROM, hard disk, or semiconductor memory. A series of processes by the system and terminals described in this specification may be realized using software, hardware, or a combination of software and hardware. It is possible to create a computer program for realizing each function of the information sharing support apparatus 10 according to this embodiment and implement it in a PC or the like. It is also possible to provide a computer-readable recording medium storing such a computer program. The recording medium may be, for example, a magnetic disk, an optical disk, a magneto-optical disk, a flash memory, etc. The computer program may also be distributed via a network, for example, without using a recording medium.

The evaluation terminal 40 according to this embodiment acquires video images from the video session service terminal, identifies at least facial images included in the video images for each predetermined frame unit, and calculates evaluation values related to the facial images (details will be described later).

### Method of Acquiring Videos

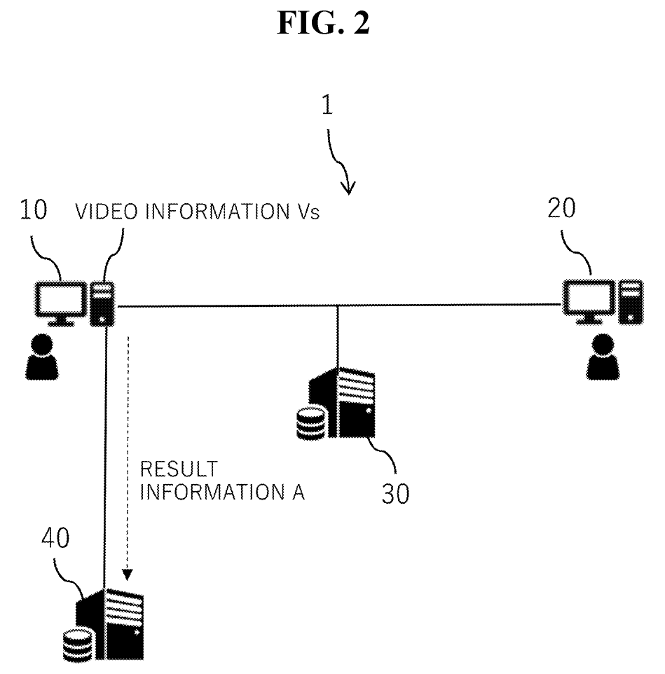

As shown in FIG. 2, the video session service provided by the video session service terminal (hereinafter may be simply referred to as "this service") enables communication between user terminals 10, 20 bidirectionally through images and sound. This service displays video images captured by the camera unit of the other user's terminal on the display of the user terminal and outputs sound captured by the microphone unit of the other user's terminal from the speaker. Also, this service is configured to allow either or both user terminals to record (recording) video images and sound (collectively referred to as "video images, etc.") in the storage unit on at least one of the user terminals. The recorded video image information Vs (hereinafter referred to as "recorded information") is cached on the user terminal that started the recording and is recorded only on the local storage of either user terminal. If necessary, users can view the recorded information themselves or share it with others within the scope of using this service.

### Analysis Flow

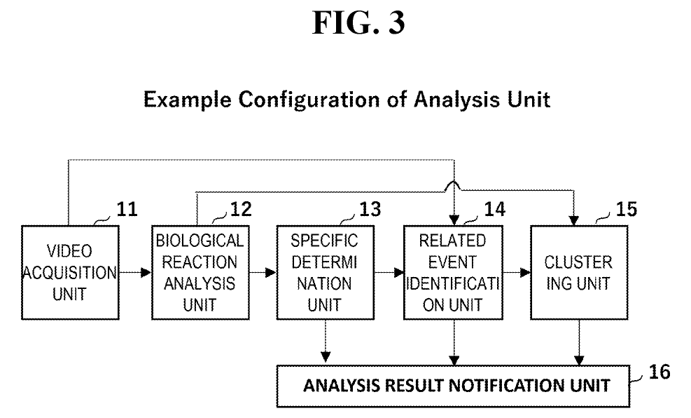

FIG. 3 is a block diagram showing a configuration example according to this embodiment. As shown in FIG. 3, the video analysis apparatus of this embodiment is realized as a functional configuration of the user terminal 10. That is, user terminal 10 includes, as its functions, a video image acquisition unit 11, a biological response analysis unit 12, a specificity determination unit 13, a related event identification unit 14, a clustering unit 15, and an analysis result notification unit 16.

The video image acquisition unit 11 acquires video images obtained by filming multiple people (multiple users) with cameras equipped in each terminal during online sessions. It does not matter whether the video images acquired from each terminal are set to be displayed on the screen of each terminal or not. That is, the video image acquisition unit 11 acquires video images from each terminal, including both video images that are currently displayed on each terminal and those that are not displayed.

The biological response analysis unit 12 analyzes changes in biological responses for each of the multiple people based on the video images acquired by the video image acquisition unit 11 (regardless of whether they are currently displayed on the screen or not). In this embodiment, the biological response analysis unit 12 separates the video images acquired by the video image acquisition unit 11 into a set of images (collection of frame images) and sound, and analyzes changes in biological responses from each.

For example, the biological response analysis unit 12 analyzes changes in biological responses related to at least one of facial expressions, eye gaze, pulse rate, and facial movements by analyzing the user's facial images using frame images separated from the video images acquired by the video image acquisition unit 11. Also, the biological response analysis unit 12 analyzes changes in biological responses related to at least one of the user's speech content and voice quality by analyzing the sound separated from the video images acquired by the video image acquisition unit 11.

When a person's emotions change, they manifest as changes in biological responses such as facial expressions, eye gaze, pulse rate, facial movements, speech content, and voice quality. In this embodiment, the user's emotional changes are analyzed by analyzing changes in the user's biological responses. The emotions analyzed in this embodiment are, for example, the degree of pleasure/displeasure. In this embodiment, the biological response analysis unit 12 calculates biological response index values that reflect the content of changes in biological responses by numerically quantifying the changes in biological responses according to predetermined criteria.

The analysis of changes in facial expressions is performed, for example, as follows. That is, for each frame image, the facial region is identified from the frame image, and the facial expression of the identified face is classified into multiple categories according to a pre-machine-learned image analysis model. Then, based on the classification results, it analyzes whether positive facial expression changes or negative facial expression changes are occurring between consecutive frame images, and how significant these facial expression changes are, and outputs a facial expression change index value according to the analysis results.

The analysis of changes in eye gaze is performed, for example, as follows. That is, for each frame image, the eye region is identified from the frame image, and by analyzing the direction of both eyes, it analyzes where the user is looking. For example, it analyzes whether the user is looking at the face of the displayed speaker, at the shared materials being displayed, or outside the screen. It may also analyze whether the eye movement is large or small, or whether the frequency of movement is high or low. Changes in eye gaze are also related to the user's level of concentration. The biological response analysis unit 12 outputs an eye gaze change index value according to the analysis results of eye gaze changes.

The analysis of changes in pulse rate is performed, for example, as follows. That is, for each frame image, the facial region is identified from the frame image. Then, using a pre-trained image analysis model that captures the numerical values of facial color information (G of RGB), it analyzes changes in the G color on the facial surface. By arranging the results along the time axis, a waveform representing changes in color information is formed, and the pulse rate is identified from this waveform. When people are tense, their pulse rate increases, and when they calm down, their pulse rate decreases. The biological response analysis unit 12 outputs a pulse rate change index value according to the analysis results of pulse rate changes.

The analysis of changes in facial movements is performed, for example, as follows. That is, for each frame image, the facial region is identified from the frame image, and by analyzing the direction of the face, it analyzes where the user is looking. For example, it analyzes whether the user is looking at the face of the displayed speaker, at the shared materials being displayed, or outside the screen. It may also analyze whether the facial movement is large or small, or whether the frequency of movement is high or low. It may also analyze the movements of the face and the eyes together. For example, it may analyze whether the user is looking straight at the face of the displayed speaker, looking up or down at it, or looking at it from an angle. The biological response analysis unit 12 outputs a facial orientation change index value according to the analysis results of changes in facial orientation.

The analysis of speech content is performed, for example, as follows. That is, the biological response analysis unit 12 converts speech into text by performing known speech recognition processing on speech for a specified time (for example, about 30-150 seconds), and by performing morphological analysis on the text, removes unnecessary words for expressing conversation, such as particles and articles. Then, the remaining words are vectorized, and it analyzes whether positive emotional changes or negative emotional changes are occurring, and how significant these emotional changes are, and outputs a speech content index value according to the analysis results.

The analysis of voice quality is performed, for example, as follows. That is, the biological response analysis unit 12 identifies the acoustic features of the voice by performing known voice analysis processing on voice for a specified time (for example, about 30-150 seconds). Then, based on these acoustic features, it analyzes whether positive voice quality changes or negative voice quality changes are occurring, and how significant these voice quality changes are, and outputs a voice quality change index value according to the analysis results.

The biological response analysis unit 12 calculates the biological response index value using at least one of the facial expression change index value, eye gaze change index value, pulse rate change index value, facial orientation change index value, speech content index value, and voice quality change index value calculated as described above. For example, it calculates the biological response index value by performing weighted calculations of the facial expression change index value, eye gaze change index value, pulse rate change index value, facial orientation change index value, speech content index value, and voice quality change index value.

The specificity determination unit 13 determines whether the changes in biological responses analyzed for the analysis target person are specific compared to the changes in biological responses analyzed for others besides the analysis target person. In this embodiment, the specificity determination unit 13 determines whether the changes in biological responses analyzed for the analysis target person are specific compared to others based on the biological response index values calculated by the biological response analysis unit 12 for each of the multiple users.

For example, the specificity determination unit 13 calculates the variance of the biological response index values calculated by the biological response analysis unit 12 for each of the multiple people, and by comparing the biological response index value calculated for the analysis target person with the variance, determines whether the changes in biological responses analyzed for the analysis target person are specific compared to others.

Three patterns can be considered for cases where the changes in biological responses analyzed for the analysis target person are specific compared to others. The first is when no particularly significant changes in biological responses have occurred for others, but relatively significant changes in biological responses have occurred for the analysis target person. The second is when no particularly significant changes in biological responses have occurred for the analysis target person, but relatively significant changes in biological responses have occurred for others. The third is when relatively significant changes in biological responses have occurred for both the analysis target person and others, but the content of the changes differs between the analysis target person and others.

The related event identification unit 14 identifies events occurring in relation to at least one of the analysis target person, others, and the environment when changes in biological responses determined to be specific by the specificity determination unit 13 occur. For example, the related event identification unit 14 identifies the analysis target person's own words and actions from the video images when specific changes in biological responses occur for the analysis target person. Also, the related event identification unit 14 identifies others' words and actions from the video images when specific changes in biological responses occur for the analysis target person. Additionally, the related event identification unit 14 identifies the environment from the video images when specific changes in biological responses occur for the analysis target person. The environment includes, for example, shared materials currently displayed on the screen, things visible in the background of the analysis target person, and so on.

The clustering unit 15 analyzes the degree of correlation between changes in biological responses determined to be specific by the specificity determination unit 13 (for example, one or a combination of multiple aspects of eye gaze, pulse rate, facial movements, speech content, voice quality) and events occurring when these specific changes in biological responses occur (events identified by the related event identification unit 14), and if the correlation is determined to be above a certain level, clusters the analysis target person or events based on the analysis results of that correlation.

For example, when specific changes in biological responses correspond to negative emotional changes, and events occurring when these specific changes in biological responses occur are also negative events, a correlation above a certain level is detected. The clustering unit 15 clusters the analysis target person or events into one of multiple pre-segmented classifications according to the content of the events, the degree of negativity, the magnitude of correlation, and so on.

Similarly, when specific changes in biological responses correspond to positive emotional changes, and events occurring when these specific changes in biological responses occur are also positive events, a correlation above a certain level is detected. The clustering unit 15 clusters the analysis target person or events into one of multiple pre-segmented classifications according to the content of the events, the degree of positivity, the magnitude of correlation, and so on.

The analysis result notification unit 16 notifies the person who designated the analysis target person (either the analysis target person or the host of the online session) of at least one of the changes in biological responses determined to be specific by the specificity determination unit 13, events identified by the related event identification unit 14, and classifications clustered by the clustering unit 15.

For example, the analysis result notification unit 16 notifies the analysis target person themselves of their own words and actions as events occurring when specific changes in biological responses different from others occur for the analysis target person (any of the three patterns mentioned above; the same applies hereafter). This allows the analysis target person to understand that they have emotions different from others when they perform certain words and actions. At this time, the specific changes in biological responses identified for the analysis target person may also be notified to the analysis target person. Furthermore, the changes in biological responses of others being compared may also be notified to the analysis target person.

For example, when the emotions received by others in response to words and actions performed by the analysis target person either routinely without particular awareness or consciously with certain emotions differ from the emotions the analysis target person themselves had at the time of the words and actions, those words and actions of the analysis target person themselves are notified to the analysis target person. This makes it possible to discover words and actions that are well-received by others or not well-received by others contrary to one's own awareness.

Also, the analysis result notification unit 16 notifies the host of the online session of events occurring when specific changes in biological responses different from others occur for the analysis target person, along with the specific changes in biological responses. This allows the host of the online session to know what kinds of events influence what kinds of emotional changes as phenomena specific to the designated analysis target person. And based on that understanding, it becomes possible to take appropriate measures for the analysis target person.

Also, the analysis result notification unit 16 notifies the host of the online session of events occurring when specific changes in biological responses different from others occur for the analysis target person or the clustering results of the analysis target person. This allows the host of the online session to understand the tendencies of behaviors specific to the analysis target person or predict possible future behaviors and states based on which classification the designated analysis target person has been clustered into. And it becomes possible to take appropriate measures for the analysis target person accordingly.

### 2. Model Generation

### Overview

The system according to this embodiment comprises a storage unit that stores a mental disorder learning model trained using training videos of patients diagnosed with mental disorders as teaching data; an acquisition unit that acquires videos containing an analysis target person; and an analysis unit that analyzes whether the analysis target person has a mental disorder by applying the mental disorder learning model to the videos.

### Model Generation Technique

The mental disorder learning model uses videos that capture the characteristics of behaviors, facial expressions, utterances, etc. of patients diagnosed with mental disorders as teaching data. This enables the model to learn the characteristics and patterns of mental disorders and acquire the ability to analyze the presence or absence of mental disorders in new video data.

In particular, the training videos according to this embodiment are recordings of both a first subject and a second subject when they are undergoing a diagnostic process for the mental disorder by an expert. Here, the first subject is a person who has been previously diagnosed with the mental disorder, and the second subject is a person who has not been previously diagnosed with the mental disorder.

The first subject is a person who has already been diagnosed with a mental disorder before the video is recorded. The videos of this subject function as the primary teaching data for the model to learn clear characteristics of mental disorders. From the videos of the first subject, the model can learn typical symptoms, reactions, facial expressions, utterances, and other characteristics of mental disorders.

The second subject is a person who has not been diagnosed with a mental disorder before the video is recorded. The videos of this subject help the model learn the characteristics of normal responses and behaviors. They are also essential for preventing overfitting and enhancing the ability to determine that there is no mental disorder by learning the characteristics of normal behaviors.

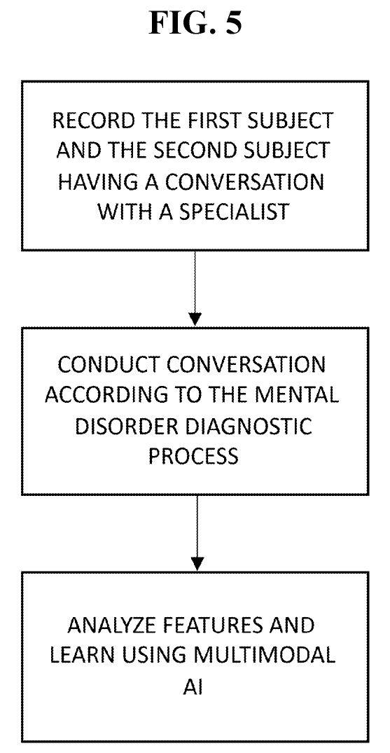

As shown in FIG. 5, the specific procedure for generating the mental disorder learning model is as follows.

### Step 1

Have the first subject and the second subject converse with an expert, and record this situation. Here, both video (visual information) and audio (auditory information) data are acquired.

Exemplifying the above-mentioned diagnostic process, the "diagnostic criteria for depression" are as follows. That is, the diagnosis of depression is based on the patient's self-reported symptoms, clinical observations, and standard diagnostic criteria (e.g., DSM-5 or ICD-10, etc.). For example, an expert conducts an interview to determine whether the following symptoms are present:

1. Depressed mood or feelings of depression

2. Loss of interest or pleasure

3. Fatigue or loss of energy

4. Feelings of worthlessness or excessive or inappropriate guilt

5. Decreased concentration or difficulty making decisions

6. Insomnia or hypersomnia

7. Weight changes associated with increased or decreased appetite 8. Thoughts of self-harm or suicidal thoughts or plans

### Step 2

More specifically, conversations between the expert and the subjects are conducted according to clinically recognized diagnostic processes for mental disorders. This process clearly captures signs and characteristics of mental disorders such as the subject's responses, facial expressions, word choices, and tone, which contribute to model generation. Through conversation with the patient, the expert may evaluate the presence or degree of the depression symptoms listed above or may label the subjects. Information about the patient's daily life and activity level, past medical history and family history, medications in use, and other treatments may also be collected.

The following examples illustrate conversations with experts that may be acquired:

1. Expert: "How have you been feeling lately? How does your mood change from day to day?"

Subject: "I've been depressed for a while and don't feel like doing anything. I feel like I can't enjoy anything."

2. Expert: "How is your sleep at night? Do you wake up several times during the night?"

Subject: "I wake up several times during the night, and in the morning, I'm tired and find it difficult to get up."

3. Expert: "Have you noticed any changes in your eating habits recently? Has your appetite increased or decreased?"

Subject: "I hardly have any appetite, and even eating feels like a chore."

4. Expert: "How do you feel about yourself? Has your sense of self-evaluation or worth changed?"

Subject: "I often feel like I can't do anything, and my sense of worthlessness has intensified."

### Step 3

Analyze the features of all subjects' conversations using multimodal AI to generate a learning model. By utilizing multimodal AI, information obtained from both video and audio can be maximally utilized to generate a high-precision mental disorder learning model.

### Other Model Generation Methods

The model according to this embodiment may be generated by the following methods:

Supervised Learning: Use a combination of videos of patients with mental disorder diagnosis results and those diagnosis results (labels). The model learns by extracting features from the videos and associating them with the labels.

Transfer Learning: Use existing video recognition models or facial recognition models as a foundation and train them on features specific to mental disorders. Use models pre-trained on large amounts of general video data and fine-tune them with small amounts of mental disorder data.

Deep Learning: Use neural networks to capture deep features of image and audio data in videos. Learn visual features in videos using CNN (Convolutional Neural Network), and features with temporal continuity using RNN (Recurrent Neural Network) or LSTM (Long Short Term Memory).

Reinforcement Learning: Based on diagnostic results, reward the model when it makes correct judgments to enable iterative learning. This allows the model to learn behaviors that produce the most accurate analysis results.

By appropriately combining these learning methods, a learning model capable of capturing the characteristics of mental disorders with high precision is generated.RetryClaude can make mistakes. Please double-check responses.

### 3. Analysis of the Analysis Target Person

### Acquisition of Videos

Record the analysis target person engaged in daily conversation.

In this embodiment, daily conversation refers to linguistic communication as a social interaction that naturally occurs among people present, rather than being conducted for a specific duty or purpose. For example, conversations in medical settings may include professional conversations for examination and diagnosis (conversations conducted as part of medical practice, such as confirming symptoms, explaining test results, discussing treatment plans) and daily conversations (casual talks in the waiting room, discussions about the weather before or after examination, chatting about family updates, natural dialogues outside the purpose of medical treatment). Daily conversations are characterized by low purposefulness (not primarily aimed at specific problem-solving or information gathering) and spontaneity (not being planned). That is, even in the same medical setting, conversations for medical interviews or diagnosis are excluded from "daily conversations," while conversations such as casual chats exchanged between examinations are included in "daily conversations." Note that daily conversations may require bidirectionality (being mutual exchanges rather than one-sided information transmission), but in this embodiment, situations where a patient is talking one-sidedly while a doctor is listening are also included in daily conversations. Daily conversations may also be recognized for their functions of maintaining and building social relationships.

Recording of daily conversations may, for example, involve recording routine online meetings. It may also serve as a setting that mimics conversations with friends or family in daily life, or dialogues with computer-generated agents. In the case of agents, they may be programmed to ask general everyday questions or questions designed to elicit deep emotions, in order to observe the reactions of the analysis target person.

### Recording on the Analysis Target Person's/Partner's Terminal

Recording of daily conversations can be performed, for example, by the user terminal 10 operated by the analysis target person. Alternatively, recording of daily conversations may be performed by the user terminal 10 operated by a partner such as the analysis target person's family member (parent, child, sibling, etc.) or friend. The user terminal 10 may be, for example, a smartphone, smartwatch, personal computer, etc., and is assumed to be equipped with a camera and microphone. On the user terminal 10, for example, video calls can be captured to obtain videos (which may include audio) in the background. The application that executes video calls may be a telephone application, a video conference application, a chat application, or any arbitrary application that is installed on all or part of the user terminal 10. Also, if an online session is conducted through the video session service terminal 30, the video data accumulated by the video session service terminal 30 may be acquired. While the analysis target person is conducting a voice call with the device away from their ear, the inline camera of the mobile terminal may capture the analysis target person's appearance during the call as still images or videos. The user terminal 10 can be set to capture these video calls constantly, periodically, at random timings, or at any timing set in advance. The user terminal 10 can send the acquired videos to the evaluation terminal 40. It should be noted that the learning model may be equipped on the user terminal 10 (the analysis target person's user terminal 10 and/or the partner's user terminal 10), allowing the user terminal 10 to perform inference on the presence or absence of mental disorders, in which case the videos can be stored in the user terminal 10 without sending them to the evaluation terminal 40.

### Recording Dialogues with Agents

Recording of daily conversations can be performed at a terminal that provides an agent (for example, the video session service terminal 30). The video session service terminal 30 can generate audio data for conversations while displaying an avatar as an agent, and play the audio data for the analysis target person. The audio data for conversations can be generated, for example, by providing a large language model with a prompt that includes text data obtained by voice recognition of the analysis target person's utterances and instructions to create conversation content in response. The video session service terminal 30 can acquire the analysis target person's conversations with the agent as video data (which can include audio data capturing utterances and video data filming the analysis target person's appearance, for example, with an inline camera).

### Recording at Medical Institutions

Small talk that takes place between a doctor and a patient (analysis target person), or between a medical professional such as a nurse and a patient, during a medical interview (before, during, or after the interview, or at least any of these times) may be filmed and acquired as videos of daily conversations. For example, a user terminal 10 operated by a medical professional can film the patient and the medical professional engaged in daily conversation and send this to the evaluation terminal 40. The user terminal 10 operated by the medical professional may be, for example, a medical terminal for viewing medical record information, or it may be a terminal different from the medical terminal. If the medical professional's user terminal 10 films the analysis target person's daily conversation, the video data may be sent from the medical professional's user terminal 10 to the evaluation terminal 40, or the medical professional's user terminal 10 may store the learning model to allow the medical professional's user terminal 10 to perform inference on the presence or absence of mental disorders based on the video data.

### Application of the Analysis Model

Input the acquired videos into multimodal AI. The multimodal AI can be a learning model trained via machine learning using video data that includes patients diagnosed with mental disorders. Additionally, the multimodal AI can be a learning model created through machine learning using training data that includes, as mentioned above, voice quality analysis results, analysis results of text converted from speech through voice recognition, detection results of biological responses such as emotions analyzed from video, and the presence or absence of mental disorders. For the analysis, information is obtained from both the video data (facial expressions, body language, etc.) and audio data (way of speaking, word choices, etc.) of the videos. Using the previously generated mental disorder learning model, it is possible to analyze the features and patterns contained in videos filming the daily conversations of the analysis target person and determine whether there are signs or characteristics of mental disorders.

### Obtaining Numerical Values

The system can include an output unit that outputs the AI analysis results as numerical data. This numerical value may, for example, be a value between 0 and 1, with values closer to 1 indicating stronger characteristics of mental disorders. This numerical value is used as an indicator of the probability or degree of mental disorder. The output unit may be equipped in the evaluation terminal 40 or in the user terminal 10.

### Setting Thresholds and Determination

Based on a pre-set threshold, determine whether the analysis target person may have a mental disorder. For example, if the numerical value is 0.7 or higher, it is determined that there are signs of mental disorder with a high probability. This threshold is set based on previous research and clinical experience and is expected to be updated according to advancements in the field and research developments. Ultimately, this system can enable early detection of mental disorders.

### Output Format

The aforementioned determination results are output in a predetermined format. Output formats can include, for example, PDF reports, notifications via email, displays on dashboards for HR cloud services, API integration, etc. Recipients of the output can include specialists such as attending physicians or counselors, the target persons themselves, family members, management departments of organizations such as companies or schools, insurance companies, rehabilitation facilities, etc.

### Output During Doctor's Medical Examination

When outputting determination results to a doctor, the system can include a result display unit that displays the determination results during the doctor's medical examination of the patient. The result display unit can display the determination results on the doctor's terminal. This allows the doctor to reference the presence or absence of mental disorders, or the possibility of mental disorders, estimated from daily conversations as a reference during diagnosis.

The doctor's terminal can obtain the presence or absence or possibility of mental disorders inferred using video data of daily conversations sent from the analysis target person's user terminal 10 to the evaluation terminal 40, from the evaluation terminal 40. Also, if inference of the presence or absence of mental disorders has been performed on the user terminal 10 of the analysis target person, partner, or medical professional, the presence or absence or possibility of mental disorders may be obtained from the user terminal 10.

The system according to this embodiment can include a consent acquisition unit that acquires consent from the analysis target person and/or partner (related person) for disclosure of the determination results to the doctor. The result display unit can display the determination results on the doctor's terminal when consent is obtained from both the analysis target person and the partner.

### Output to the Analysis Target Person

Output according to the determination results can be provided from the analysis target person's user terminal 10 (such as a smartphone, etc.).

### Messages Showing Concern

For example, the output unit can output messages showing concern to the analysis target person according to the determination results. Messages showing concern can be, for example, inquiry messages such as "Is everything okay?" Messages from the output unit may be displayed as text on the analysis target person's user terminal 10, or audio data of the messages processed by voice synthesis may be played on the user terminal 10.

### Smart Home Control

For example, the output unit can include a control unit that controls devices placed in the analysis target person's residence according to the determination results. Devices can include, for example, lighting fixtures, music playback devices such as smart speakers, display devices such as televisions or monitors, etc.

The control unit can control lighting fixtures to have different brightness levels according to the determination results. For example, if the analysis target person is determined to have a mental disorder, or if the possibility of having a mental disorder is above a predetermined value, the control unit can turn on lighting fixtures or increase the brightness of lighting fixtures.

The control unit can, for example, control devices such as smart speakers or music players on personal computers to play different songs according to the determination results. For example, if the analysis target person is determined to have a mental disorder, or if the possibility of having a mental disorder is above a predetermined value, the control unit can select and play up-tempo songs (for example, songs with BPM (Beats Per Minute) greater than a predetermined threshold).

### Photo Display

The control unit can control the display of different photos according to the determination results. Photo data is assumed to be stored in a predetermined storage unit (photo data storage unit). Also, the display history of photos viewed by the analysis target person is assumed to be stored in a predetermined storage unit (display history storage unit). The photo data storage unit and display history storage unit may be equipped in the user terminal 10, or they may be equipped in the evaluation terminal 40 with the user terminal 10 having access to the photo data. The control unit can display different photos on display devices (which may be mobile terminals such as smartphones, or display devices such as monitors or smart TVs) according to the determination results. In doing so, the control unit can select photos to display based on the display history of photo data. For example, if the analysis target person is determined to have a mental disorder, or if the possibility of having a mental disorder is above a predetermined value, the control unit can display photo data that the analysis target person frequently views. Whether the analysis target person frequently views certain photos can be determined by whether the number of views is above a predetermined number (which may be an average viewing count or a fixed value). The number of views may count the display count for a specific period (which may be the entire period, the most recent period, or for each past specified long period (e.g., one year from the present, the year viewed, fiscal year, etc.)).

Also, if it is possible to film the analysis target person viewing photo data on the user terminal 10, the analysis unit can analyze those images (videos or still images) to detect biological responses such as emotions. The control unit can aggregate the number of times a given positive emotion was detected during viewing or the viewing time during which the person was viewing with a given positive emotion, and can identify photo data that is expected to evoke positive emotions during viewing based on these aggregate values (count, time, or a combination of these). The control unit can control the display to show the identified photo data that is expected to evoke positive emotions during viewing.

### Call Suggestion

The output unit can suggest calls to different call partners according to the determination results. For example, the system can be equipped with a call history storage unit that stores the analysis target person's call history. The call history includes information identifying multiple callers (at least two parties) and the date and time of calls. The output unit can search for call history containing information identifying the analysis target person. The output unit can identify the analysis target person's call partners from the searched call history and aggregate the number of calls and/or call duration for each call partner. Aggregation of the number of calls and/or call duration may be performed for specific periods (which may be the entire period, the most recent period, or for each past specified long period (e.g., one year from the present, the year viewed, fiscal year, etc.)). If the analysis target person is determined to have a mental disorder, or if the possibility of having a mental disorder is above a predetermined value, the output unit can identify call partners with whom the analysis target person has had calls a predetermined number of times or more, and/or for a predetermined duration or longer, and suggest calls with the identified call partners.

Also, if it was possible to film the analysis target person during calls on the user terminal 10 (or if calls were conducted via online sessions and video data could be obtained from the video session service terminal 30), the analysis unit can analyze those images (videos or still images) to detect biological responses such as emotions. The output unit can aggregate the number of times a given positive emotion was detected during calls or the call duration during which the person was on a call with a given positive emotion, and can identify call partners with whom positive emotions are expected during calls based on these aggregate values (count, time, or a combination of these). The output unit can suggest calls with the identified call partners with whom positive emotions are expected during calls.

Also, by including call content (which may be video data or audio data, or text data of call content obtained through voice analysis) in the call history, the analysis unit can determine the analysis target person's stress level during the call as a biological response. The analysis unit can aggregate the number of times the stress level exceeded a predetermined value during calls, or the duration for which the stress level exceeded a predetermined value, and can identify call partners who cause stress to the analysis target person during calls based on these aggregate values (count, time, or a combination of these). The output unit can suggest call partners by excluding those identified as causing stress to the analysis target person during calls from among the analysis target person's call partners.

### Action Suggestion

The output unit can include a decision unit that determines actions that the analysis target person should take according to the determination results. The output unit can suggest actions determined by the decision unit to the analysis target person (for example, displaying them as text or notifying by voice). For example, when having a conversation with an avatar, actions can be suggested as utterances from the avatar.

The system can include an action storage unit that stores actions associated with candidate determination results. The decision unit can retrieve actions corresponding to the determination results from the action storage unit.

Also, the decision unit can generate actions by providing a large language model with a prompt that includes instructions to generate actions that should be taken by a person with the determination results. Additionally, by providing a large language model with a prompt that includes multiple actions stored in the action storage unit, the determination results, and instructions to select one or more from the multiple actions based on the determination results, one or more actions can be selected. The output unit can suggest the selected actions to the analysis target person.

### Analysis Target Examples

The diseases targeted by this system can include, for example, the following: depression, bipolar disorder, schizophrenia, generalized anxiety disorder, obsessive-compulsive disorder, panic disorder, social anxiety disorder, PTSD, borderline personality disorder, antisocial personality disorder, dependent personality disorder, avoidant personality disorder, attention deficit hyperactivity disorder, autism spectrum disorder, alcohol use disorder, drug use disorder, Alzheimer's dementia, Lewy body dementia, vascular dementia, anorexia nervosa, bulimia nervosa, insomnia, sleep apnea syndrome, hypoactive sexual desire disorder, sexual dysfunction, conversion disorder, dissociative disorder, somatoform disorder, factitious disorder imposed on another, factitious disorder imposed on self.

The processes described in this specification using flowchart diagrams need not necessarily be executed in the order shown. Some process steps may be executed in parallel. Additionally, additional process steps may be employed, or some process steps may be omitted.

The embodiments described above may be implemented in appropriate combinations. Furthermore, the effects described in this specification are merely explanatory or exemplary and not limiting. That is, the technology according to this disclosure may, along with the above effects or instead of the above effects, produce other effects that are apparent to those skilled in the art from the description in this specification.

### Disclosure Items

The disclosure in this specification also includes the following items.

### Item 1: Displaying results from daily conversations during medical examination

### [Item 1-1] (Determining the presence or absence of mental disorders from daily conversations and displaying it on the doctor's terminal during medical examination)

An information processing system comprising:

a storage unit that stores a mental disorder learning model trained using training videos of patients diagnosed with mental disorders as teaching data;

an acquisition unit that acquires an analysis target video related to daily conversations of an analysis target person;

a determination unit that determines whether the analysis target person has the mental disorder by inputting the analysis target video into the mental disorder learning model; and

a result display unit that displays the determination result of whether the analysis target person has the mental disorder on a terminal used by a doctor during medical examination of the analysis target person.

### Item 2: Determining the presence or absence of mental disorders in an analysis target person from conversations between the partner (spouse, parent, etc.) and the analysis target person on the partner's side

### [Item 2-1] (The analysis target person's partner understands the partner's mental state)

An information processing system comprising:

a storage unit that stores a mental disorder learning model trained using training videos of patients diagnosed with mental disorders as teaching data;

an acquisition unit that acquires an analysis target video, which is video data of a video call containing daily conversations between a first terminal of a person related to the analysis target person and a second terminal of the analysis target person; and

a determination unit that determines whether the analysis target person has the mental disorder by inputting the analysis target video into the mental disorder learning model.

### [Item 2-2] (Combination with Item 1)

The information processing system according to Item 2-1, comprising:

a storage unit that stores a determination result by the determination unit; and

a result display unit that displays the determination result of whether the analysis target person has the mental disorder on a terminal used by a doctor during medical examination of the analysis target person.

### [Item 2-3]

The information processing system according to Item 2-1 or Item 2-2, comprising:

a determination result storage unit that stores a determination result by the determination unit; and

a consent acquisition unit that acquires consent from the analysis target person and the person related to the analysis target person for disclosure of the determination result to the doctor,

wherein the result display unit displays the determination result on the terminal when consent is obtained from both the analysis target person and the person related to the analysis target person.

### Item 3: Determining the presence or absence of mental disorders in an analysis target person from conversations conducted using the analysis target person's mobile terminal (such as a smartphone)

### [Item 3-1] (Understanding the mental state of the analysis target person on the analysis target person's mobile terminal)

An information processing system comprising:

a storage unit that stores a mental disorder learning model trained using training videos of patients diagnosed with mental disorders as teaching data;

an acquisition unit that acquires an analysis target video, which is video data of a video call related to daily conversations that the analysis target person is conducting on the analysis target person's terminal; and

a determination unit that determines whether the analysis target person has the mental disorder by inputting the analysis target video into the mental disorder learning model.

### [Item 3-2] (Combination with Item 5, such as "Is everything okay?")

The information processing system according to Item 3-1, comprising:

an output unit that outputs messages showing concern to the analysis target person according to the determination result by the determination unit.

### [Item 3-3] (Combination with Item 5, Smart Home)

The information processing system according to Item 3-1 or Item 3-2, comprising:

a control unit that controls devices placed in the analysis target person's residence according to the determination result by the determination unit.

### [Item 3-4] (Increasing lighting)

The information processing system according to Item 3-3, wherein

the control unit adjusts the brightness of lighting fixtures according to the determination result.

### [Item 3-5] (Playing up-tempo music)

The information processing system according to Item 3-3 or Item 3-4, wherein

the control unit plays different music according to the determination result.

### [Item 3-6] (Combination with Item 5, photo display)

The information processing system according to any one of Items 3-1 to 3-5, comprising:

a photo storage unit that stores photo data;

a display history storage unit that stores the display history of the photo data; and

a photo display unit that displays photos that the analysis target person has viewed at a predetermined frequency or higher based on the display history, according to the determination result by the determination unit.

### [Item 3-7] (Combination with Item 5, recommending phone calls)

The information processing system according to any one of Items 3-1 to 3-6, comprising:

a call history storage unit that stores the analysis target person's call history including call partners; and

a suggestion unit that suggests calls to call partners with whom the analysis target person has had calls at a predetermined frequency or higher based on the call history, according to the determination result by the determination unit.

### [Item 3-8] (Identification of stressors)

The information processing system according to Item 3-7, wherein

the call history includes call content, and

the system comprises a stress determination unit that determines the analysis target person's stress level by analyzing the call content.

### [Item 3-9] (Combination with Item 1)

The information processing system according to any one of Items 3-1 to 3-8, comprising:

a result display unit that displays the determination result of whether the analysis target person has the mental disorder on a terminal used by a doctor during medical examination of the analysis target person.

### Item 4: Conversation with AI Avatar

### [Item 4-1] (Understanding the mental state of the analysis target person from conversations with an AI avatar)

An information processing system comprising:

a storage unit that stores a mental disorder learning model trained using training videos of patients diagnosed with mental disorders as teaching data;

a conversation processing unit that conducts daily conversations via video calls with the analysis target person;

an acquisition unit that acquires an analysis target video, which is video data related to the video calls; and

a determination unit that determines whether the analysis target person has the mental disorder by inputting the analysis target video into the mental disorder learning model.

### [Item 4-2] (Recommending the next action)

The information processing system according to Item 4-1, comprising:

a decision unit that determines actions that the analysis target person should take next according to the determination result by the determination unit; and

a notification unit that notifies the analysis target person of the actions.

### [Item 4-3] (Having a LLM decide the actions)

The information processing system according to Item 4-2, wherein

the decision unit generates the actions by providing a large language model with a prompt that includes the determination result and instructions to generate actions that should be taken by a person with the determination result.

### [Item 4-4] (Listing actions in advance)

The information processing system according to Item 4-2, comprising:

an action storage unit that stores the actions associated with candidate determination results, wherein

the decision unit retrieves the actions corresponding to the determination result from the action storage unit.

### [Item 4-5] (Listing actions in advance and having a LLM select from them)

The information processing system according to Item 4-3, comprising:

an action storage unit that stores multiple actions, wherein

the decision unit has the large language model select the actions by providing it with a prompt that includes the multiple actions, the determination result, and instructions to select one or more from the multiple actions based on the determination result.

### [Item 4-6] (Combination with Item 5, Smart Home)

The information processing system according to any one of Items 4-1 to 4-5, comprising:

a control unit that controls devices placed in the analysis target person's residence according to the determination result by the determination unit.

### [Item 4-7] (Increasing lighting)

The information processing system according to Item 4-6, wherein

the control unit adjusts the brightness of lighting fixtures according to the determination result.

### [Item 4-8] (Playing up-tempo music)

The information processing system according to Item 4-6 or Item 4-7, wherein

the control unit plays different music according to the determination result.

### [Item 4-9] (Combination with Item 5, photo display)

The information processing system according to any one of Items 4-1 to 4-8, comprising:

a photo storage unit that stores photo data;

a display history storage unit that stores the display history of the photo data; and

a photo display unit that displays photos that the analysis target person has viewed at a predetermined frequency or higher based on the display history, according to the determination result by the determination unit.

### [Item 4-10] (Combination with Item 5, recommending phone calls)

The information processing system according to any one of Items 4-1 to 4-9, comprising:

a call history storage unit that stores the analysis target person's call history including call partners; and

a suggestion unit that suggests calls to call partners with whom the analysis target person has had calls at a predetermined frequency or higher based on the call history, according to the determination result by the determination unit.

### [Item 4-11] (Combination with Item 1)

The information processing system according to any one of Items 4-1 to 4-10, comprising:

a result display unit that displays the determination result of whether the analysis target person has the mental disorder on a terminal used by a doctor during medical examination of the analysis target person.

### Item 5: Light notification (providing light notification when determining mental disorders)

### [Item 5-1] (Showing concern, such as "Is everything okay?")

An information processing system comprising:

a storage unit that stores a mental disorder learning model trained using training videos of patients diagnosed with mental disorders as teaching data;

an acquisition unit that acquires an analysis target video related to daily conversations of an analysis target person;

a determination unit that determines whether the analysis target person has the mental disorder by inputting the analysis target video into the mental disorder learning model; and

an output unit that outputs messages showing concern to the analysis target person according to the determination result by the determination unit.

### [Item 5-2] (Smart Home)

An information processing system comprising:

a storage unit that stores a mental disorder learning model trained using training videos of patients diagnosed with mental disorders as teaching data;

an acquisition unit that acquires an analysis target video related to daily conversations of an analysis target person;

a determination unit that determines whether the analysis target person has the mental disorder by inputting the analysis target video into the mental disorder learning model; and

a control unit that controls devices placed in the analysis target person's residence according to the determination result by the determination unit.

### [Item 5-3] (Increasing lighting)

The information processing system according to Item 5-2, wherein

the control unit adjusts the brightness of lighting fixtures according to the determination result.

### [Item 5-4] (Playing up-tempo music)

The information processing system according to Item 3-2 or Item 5-3, wherein

the control unit plays different music according to the determination result.

### [Item 5-5] (Photo display)

The information processing system according to any one of Items 5-1 to 5-4, comprising:

a photo storage unit that stores photo data;

a display history storage unit that stores the display history of the photo data; and

a photo display unit that displays photos that the analysis target person has viewed at a predetermined frequency or higher based on the display history, according to the determination result by the determination unit.

### [Item 5-6] (Recommending phone calls)

The information processing system according to any one of Items 5-1 to 5-5, comprising:

a call history storage unit that stores the analysis target person's call history including call partners; and

a suggestion unit that suggests calls to call partners with whom the analysis target person has had calls at a predetermined frequency or higher based on the call history, according to the determination result by the determination unit.

### [Item 5-7] (Identification of stressors)

The information processing system according to Item 5-6, wherein

the call history includes call content, and

the system comprises a stress determination unit that determines the analysis target person's stress level by analyzing the call content.

1. An information processing system comprising:
a storage unit that stores a mental disorder learning model trained using training videos of patients diagnosed with mental disorders as teaching data;
an acquisition unit that acquires an analysis target video, which is video data of a video call containing daily conversation between a first terminal of a person related to an analysis target person and a second terminal of the analysis target person; and
a determination unit that determines whether the analysis target person has the mental disorder by inputting the analysis target video into the mental disorder learning model.

2. The information processing system according to claim 1, comprising:
a storage unit that stores a determination result by the determination unit; and
a result display unit that displays the determination result of whether the analysis target person has the mental disorder on a terminal used by a doctor during medical examination of the analysis target person.

3. The information processing system according to claim 2, comprising:
a determination result storage unit that stores a determination result by the determination unit; and
a consent acquisition unit that acquires consent from the analysis target person and the person related to the analysis target person for disclosure of the determination result to the doctor,
wherein the result display unit displays the determination result on the terminal when consent is obtained from both the analysis target person and the person related to the analysis target person.

4. A method executed by a computer, the method comprising:
storing a mental disorder learning model trained using training videos of patients diagnosed with mental disorders as teaching data;
acquiring an analysis target video, which is video data of a video call containing daily conversation between a first terminal of a person related to an analysis target person and a second terminal of the analysis target person; and
determining whether the analysis target person has the mental disorder by inputting the analysis target video into the mental disorder learning model.
